(19) United States
(12) Patent Application Publication
(10) Pub. No.: US 2007/0191819 A1
(43) Pub. Date: Aug. 16, 2007

(54) Cell processor for use in the treatment of diseases

(75) Inventors: Ute Steinfeld, St Ingbert (DE); Hyeck-Hee Lee, St Ingbert (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: KIST-EUROPE FORSCHUNGSGESELLSCHAFT MBH, SAARBRICLEM (DE)

(21) Appl. No.: 10/570,004

(22) PCT Filed: Aug. 23, 2004
(86) PCT No.: PCT/EP04/09414

§ 371 Date: Sep. 11, 2006

(30) Foreign Application Priority Data
Aug. 29, 2003 (DE) 103 39 905.4

(51) Int. Cl. A61K 9/22

(52) U.S. Cl. 604/891.1; 435/285.1; 435/288.5

(57) ABSTRACT

The present invention relates to a micromedical cell processor which modifies the cellular components of blood, especially cells of the body's natural immune defense, such that the cells, after their introduction into the body, exert a therapeutic function against cancerous diseases or against other diseases. The cell processor is implantable in the human or animal body and has a device for isolating cells, a device for fixing the cells, a device for introducing substances into the cells, and a device for determining the concentration of substances in the cells. The cell processor can also be used outside the body for loading the cells.

The present invention relates to a device, in the following also designated as a medical cell processor or simply a cell processor, which modifies cellular components of human or animal blood, especially cells of the body's natural immune defense, such as, for example, T-cells or macrophages, such that the cells exert a therapeutic effect, for example, against cancerous diseases, e.g., of the liver or the brain, or against other diseases, after their introduction into the human or animal body. Here and in the following sections, the cellular components described are also designated in short as cells. The cells can also involve, for example, other already differentiated, naturally occurring cells of the human or animal body or not-yet differentiated cells, such as stem cells. The cell processor can be implanted into the human or animal body, but non-implantable configurations are also possible. The therapeutic effect of the modified cells introduced into the human or animal body can involve, for example, a controlled active ingredient release or tissue regeneration or the like.

Methods for medical treatment through active ingredients have been known for a long time. In these methods, the active ingredient is usually delivered to the whole human or animal body. The active ingredient can be administered, for example, orally or by injection; it then distributes itself uniformly in the whole human or animal organism. The decisive disadvantage of the previous treatment methods is to be seen in that unaffected regions of the human or animal body can also be affected by the active ingredients and that only a small part of the active ingredient can act in the target regions. Thus, correspondingly high active ingredient doses are unavoidable.

The task of the present invention is to make available a device, for example, a device that can be implanted into the human or animal body and that permits human or animal cells to be modified such that these cells are delivered to targeted, desired body parts or cells after being introduced again into the human or animal body, and there exert a therapeutic effect. By using a device configured in this way it is thus possible, for example, to fight diseases with low dosing in a targeted way or to build up and strengthen tissue in a targeted way, without affecting uninvolved regions of the body.

This problem is solved by a device according to claim 1 and also by a method according to claims 69 and 70. Advantageous improvements of the device according to the invention and also of the described method are described in the dependent claims.

A device or cell processor according to the invention has the following components: a device for isolating cells, for example, from the bloodstream or a blood sample, a cell line or cell culture (for example, freshly isolated cells from patients, primary cultures, etc.), advantageously a device for fixing cells, a device for introducing substances, for example, active ingredients, into the fixed cells or for attaching these substances to the fixed cells, and advantageously also a device for determining the concentration of substances in or on the cells. Advantageously, the device also has a device for introducing cells into the human or animal bloodstream. Advantageously, the cells or the cells and the medium surrounding the cells are transported between the individual devices of the cell processor, for example, with the help of micropumps integrated into or attached to the cell processor. The individual component devices for manipulating the cells are advantageously embodied as devices that are as low contact as possible, because mechanical contact between an immune cell and a surface can trigger an immune reaction.

In one embodiment, the cell processor is implanted in the direct vicinity of an artery (however, the processor can also be arranged or carried outside of the body). The bioprocessor removes blood from the bloodstream. In the chip or the processor, certain blood cells, e.g., leukocytes, are selected and these cells can also be loaded with an unencapsulated medicine. For the use of encapsulated medicines, the capsule can be composed of, e.g., thermally soluble materials, so that by heating in the target region (for example, through hyperthermia therapy), the medicine is released. The encapsulation can also be realized with the help of material that can be split by a cell's own enzymes (for example, dextran). In this case, the medicine is released after a foreseeable time span (equivalent to the use of unencapsulated medicines). For the use of unencapsulated medicines, the carrier cell itself can be destroyed by the medicine after a definable time period. Here, the cell membrane becomes permeable to the medicine. Here, the defined time period can be selected (e.g., by the type of medicine, the medicine's formulation, by the selection of the cytotoxic properties, and/or the concentration), such that the cell can be led to the target location within this time. In this way, the medicine is released in a targeted way at the target location.

The cells are released back into the bloodstream and transport the active ingredient specific to the target to the disease location, e.g., a tumor. The system or the cell processor is thus implanted in the body or carried on the body or arranged outside of the body. For modifying animal or human cells, especially for therapeutic purposes, the cell processor can be applied or used outside of the body, for example, in the laboratory as a laboratory instrument.

If the device is embodied as a device that can be used outside of the human or animal body, then, on one hand, it can be realized as a system that can be carried on the body. However, as described, the device can also be embodied as a laboratory system or as a system to be operated in the laboratory for modifying human or animal cells, for example, for therapeutic purposes. One example is a laboratory system for modifying cells for autologous cancer therapies, in which, for example, immune cells are isolated from the tumor of a patient, then propagated in the laboratory, and finally released back into the bloodstream of the patient. These cells can then be loaded, after the propagation step in the laboratory, with nanoparticles which are coated or provided with medicine and/or nucleic acids and/or other therapeutically active compounds with the device according to the invention. Applications of the laboratory device for screening purposes for pharmaceutically active ingredients are also conceivable.

Thus, the cell processor can be implantable, but can also be provided in a non-implantable form and/or size. The cell treatment, thus, for example, the isolation, fixing, transporting, or counting of cells, etc., however, can also be realized with contact. One example here is cell sorting by means of antibody binding.

The first component device of the cell processor designed according to the invention is the device for isolating the cells. In a first advantageous configuration, this isolating device is composed of at least one capillary, for example, made from a high-polymer plastic, as well as at least two electrodes arranged on this capillary or these capillaries. The cells are led through the capillary, wherein the capillary diameter is designed so that cells can only pass one at a time through the capillary. The individual cell types have different electrical conductivity values. If the cells contact the electrodes then, depending on the cell type, currents of different magnitudes are generated, which can be measured. In this way, the cells are distinguishable from each other and can be selected. An isolating device configured in this way thus isolates the necessary cells from their environment, for example, a blood sample, by comparing the conductivity values of different cell types. In another advantageous embodiment, at least one laser detector is arranged on the capillary or capillaries. Because the different cell types also feature different light-refracting properties, the necessary cells can be selected or isolated with reference to light refraction. In another advantageous embodiment, the necessary cells are isolated by measuring the impedance, in which different cell types also differ. In another advantageous embodiment, a widening is attached at the end at least one of the capillaries, wherein an electromagnetic field is generated on or in this widening, for example, with the help of two electrodes arranged on it. Because different cell types have particle charges with different magnitudes, they are affected by the electromagnetic field to different degrees and accordingly, have a propagation time of different lengths until reaching the capillary wall in the widened area in the electromagnetic field. In this way, the necessary cells can be isolated with the help of their particle charge. In another advantageous configuration of the device for cell isolation, the electrophoretic mobility of the cells is used. This electrophoretic mobility assumes different values depending on the cell properties, such as the density of the surface charge, volume, and weight, and therefore can also be used for selecting the desired cell type. Another advantageous configuration of the device for cell isolation uses the different particle sizes of different cell types. For this purpose, for example, a filter membrane is used which is designed so that only certain blood cells can pass through the membrane, while other blood cells are held back. Here, membrane filters with different pore sizes can also be used. Another possible configuration of the isolating device uses so-called solution diffusion membranes. Mass transfer or isolation of the desired cell type is generated or performed here, for example, using the following means and method: on the primary side of a solution diffusion membrane, a solvent is used which corresponds to all of the contents or all of the present cell types. On the secondary side of the solution diffusion membrane, a solvent is used which is suitable only for one certain component or one certain cell type. In the solvent of the secondary side, thus only the cell type to be isolated is soluble. The component or the corresponding cell type soluble on the secondary side of the solvent membrane has the tendency to diffuse through the membrane, while none of the other components or cell types have this tendency. Thus, the necessary cell type can be isolated. Obviously, other membrane types can be used for cell isolation by means of filtration. The filtration can be realized, for example, in an H-shaped filter module.

As a second component device, the cell processor according to the invention advantageously has a device for fixing the cells. The fixing is used here for holding the cells, so that the substance or the active ingredient can be added to or introduced into the cells. A first advantageous configuration of the component device for cell fixing is composed of a capillary, for example, a high-polymer plastic, a widening at the end of this capillary, and a device inserted into the widening for holding the cells. The device for holding the cells is, for example, a fine needle, to which an electric field is applied. The device uses the particle charges of the cells. However, the maintenance of the particle charge assumes a certain amount of movement of the cells. Therefore, in the present case, an alternating electric field is used, which alternately holds the cells tight and then pushes them away again, so that these remain in motion and the particle charge is thus maintained. The cells to be fixed are thus held by an alternating electric field. Such a component device for fixing the cells with the help of an electric field can be configured, for example, in the form of a three-dimensional microelectrode system for contactless cell manipulation. Such a three-dimensional microelectrode system enables the fixing or the holding of cells in a cage filled with a dielectric liquid. Here, the three-dimensional microelectrode system has, for example, the following components.
a two-layer electrode structure or two electrodes which are separated by a 40-μm-thick flow channel made from high-polymer plastic.
the electrode elements, which can have the shape of a shaft, hose, or funnel, or even the shape of a cage or a switch, are operated by an alternating current or a rotating electric field.
the electrode thickness equals 10 μm and the active electrode surfaces are minimized, in order to prevent the heating of the solution.
the system is operated at 5-11 V and at 5-15 MHz.
the channel has a flow rate of 300 μm/s.

Another advantageous configuration of the component device for fixing immune defense cells is configured as follows. With the help, for example, of a fine capillary, gas or a fluid is introduced, for example, into a microball. In this way, the microball is inflated and thus simulates a foreign body. After the defense cell has surrounded the simulated foreign body, thus being fixed, the active ingredient is introduced to the cell. Then the gas or the fluid is released from the microball and the defense cell swims free again. This defense cell fixing through foreign-body simulation thus uses the natural function of defense cells.

A first advantageous configuration of the component device of the cell processor for introducing a substance into the cell or for arranging a substance on the cell, wherein this substance can be an active ingredient, is composed of a device for generating high-voltage pulses, for example, with the help of microelectrodes which have been coated, for example, through sputtering of gold. With the help of such high-voltage pulses, which can be realized, for example, in the form of a step-shaped potential, small reversible pores are formed in the cell membrane. Then the corresponding substances or active ingredients are introduced into the cells through these pores. In the present configuration, the active ingredient or the substance is thus introduced by means of electroporation. This also applies for all of the methods presented below for introducing a substance into the cells or for arranging a substance on the cells. The surface of the substances or active ingredients can be modified so that the substances or the active ingredients are no longer recognized by the cells.

Another advantageous configuration of the component device for introducing or arranging substances or active ingredients uses magnetizable or magnetized nanoparticles or small spherules, which are coated with or contain the substance or the active ingredient. Here, nanoparticles are usually particles with a size of a few nanometers up to a few hundred nanometers. However, below, particles with a size, for example, in the micrometer range are also to be understood as nanometers. The component device now contains a device for generating a magnetic field. This can contain or be composed of, for example, at least one microcoil. The particles or spherules are set in motion or vibration with the help of this magnetic field, which here advantageously concerns an oscillating field, but static fields are also possible, and therefore tends to penetrate into the cell at a sufficient field strength of the magnetic field. The oscillating field can be, for example, sinusoidal-shaped or sawtooth-shaped. If the particles or spherules are found in simple liquids, then preferably static magnetic fields are used. If they are found in more complex biological media, then preferably oscillating fields are used. An advantageous configuration of the magnetic field-based introduction device here has a reservoir which is filled with the corresponding nanoparticles or spherules. A capillary with a lock is attached to the reservoir, wherein the lock always lets pass only a precisely defined number of nanoparticles or spherules. A magnet is mounted underneath the capillary. The cell to be modified is fixed between the magnet and the capillary. The substances or active ingredients are added to the corresponding nanoparticles or spherules. These are optionally still magnetized before or after. If the magnet in this arrangement is activated, then the nanoparticles or spherules are pulled into the cell. The decisive factors are the time and the strength of the magnetic field: the nanoparticles or spherules may not completely pass through the cell, they must remain in it. The nanoparticles or spherules can be, for example, particles containing Fe₂O₃ or Fe₃O₄ or paramagnetic particles. The cells loaded with the nanoparticles using the described means and methods can also be used for diagnostic purposes (comparable to the application of contrast means). The nanoparticles can also be coated with contrast means or similar materials of high atomic number. The cells loaded with nanoparticles can then also help through the physical properties of the nanoparticles, such as, for example, magnetic characteristics (magnetized nanoparticles), for disease identification or better recognizability of diseases. The cells loaded with nanoparticles can also be used for diagnoses and simultaneously for medication. This is then possible through the magnetic properties of the nanoparticles and their simultaneous coating with an active ingredient.

In another advantageous configuration of the component device for introducing substances or active ingredients into the cells, liposomes are used. Liposomes have the ability to penetrate the cell membrane of a cell, thus with their help a substance or an active ingredient can be transmitted into the cell. The substance or the active ingredient is thus first introduced into a liposome. This is realized advantageously as previously described via active ingredient-coated, magnetized particles. Then the substance or the active ingredient is transmitted into the cell by means of lipofection, i.e., the liposome complex fuses with the cell membrane and discharges the substance or the active ingredient into the cell. In another advantageous configuration of the component device of the cell processor for introducing a substance or an active ingredient into the cell, phagocytosis is used. Phagocytes are cells that ingest other cells. They engulf foreign matter and dissolve and destroy it by means of enzymes. Thus, the natural function of immune cells is used, in that the active ingredient or the substance is recognized as a foreign body and surrounded by an immune cell. For this purpose, the substance or the active ingredient is added in a form that makes it indigestible for the immune cell. In another advantageous configuration of the component device for introducing substances or active ingredients into the cell, viruses are used. These viruses can involve, for example, modified HIV viruses. Alternatively, DNA which triggers the cell itself to produce a desired active ingredient or a desired substance can be introduced into the cell. In a last example configuration of a component device for introducing substances or active ingredients into a cell, a very fine needle is used for microinjection. The substance or the active ingredient is injected with the help of this very fine needle through a very fine hole in the fixed cell. Instead of the needle, nanofibers made from, for example, carbon compounds can also be used for injection. Here, the nanofibers advantageously have a diameter at the tip of a few tens of nanometers. If these fibers are arranged with a distance from each other corresponding to the cell size, for example, on a silicon chip in a two-dimensional matrix, cells deposited on the chip, for example, with the help of centrifugal forces, are pierced by only one fiber, wherein a substance or an active ingredient can be injected into the cell.

A first advantageous configuration of a component device for determining the concentration of a substance or an active ingredient in or on the cell uses at least one sensor for determining magnetic field strengths. With the help of such a magnetic field sensor, upon the use of magnetized nanoparticles or spherules, the substance or the active ingredient concentration is measured in the cell by measuring the magnetic field strength of the nanoparticles or spherules. The sensitivity of the sensor is here configured advantageously according to the minimum substance or active ingredient charging, the measurement resolution according to a substance or active ingredient charge unit. In the case of the use of magnetized particles in liposomes, the sensor can also be used, for example, to determine the number of particles loaded into a liposome. In an embodiment, the magnetic field sensor contains a Hall sensor or a two-dimensional array of Hall sensor elements comprising, for example, 4×4=16 individual Hall elements. In another embodiment, the sensor involves a magnetoresistive sensor or an arrangement of microcoils which detect the magnetic fields inductively. The magnetic field sensors here work contact-free, i.e., the substance or the active ingredient concentration is determined without contacting the substance or the active ingredient coupled to the magnetic materials or the cells. In another advantageous configuration, the active ingredient or substance concentration is determined with the help of a device for measuring the fluorescent light from fluorescing pigments and/or with the help of biomarkers. For this purpose, a fluorescing substance or a marker substance is applied to the substance or to the active ingredient. In another advantageous configuration, the cell processor has a device for determining the number or for controlling the number of modified cells.

In an advantageous configuration of the cell processor according to the invention, the component device for determining the concentration of a substance or an active ingredient in or on the cell and the component device for introducing substances or active ingredients into the cell are housed in a common reaction chamber. Advantageously, this reaction chamber has at least two feed devices, such as, for example, microchannels: one feed device for feeding the cells and one feed device for feeding substances or active ingredients, optionally also washing reagents. The washing reagents are fed after the active ingredient treatment. Advantageously, the reaction chamber has other elements for the electrophoresis, such as, for example, funnel-shaped or shaft-shaped microelectrodes for aligning electrostatically charged cells or straight or zigzag-shaped microelectrodes for deflecting electrostatically charged cells.

However, the component device for determining the concentration of a substance or an active ingredient and the component device for introducing substances or active ingredients into the cell can also be arranged in different compartments (for example, a chip with different reaction chambers or compartments, so that the concentration is determined and the active ingredient is introduced in different areas of the chip). Compartments or reaction chambers are areas of the device which are separated from each other (for example, through suitable wall structures) and which can represent or integrate different functional units of the device (for example, mixing chamber, electroporation unit or separation unit). The individual compartments or reaction chambers can then be connected to each other via suitable flow channels so that the cells can be led from one compartment or reaction chamber to another.

In another advantageous configuration, the cell processor has a component device for introducing the cells into the human or animal bloodstream. This can contain, for example, micropumps, microvalves, micronozzles, and/or microfilters for controlling the flow of a fluid containing cells. If the modified cells are introduced into the human or animal body, they are then transported to a desired, defined tissue type via the bloodstream. The desired, defined tissue is reached by the ability of cells to track down this tissue with the help of messenger materials which are screened by the desired tissue. Analogously, it is also possible to discover previously unknown diseased tissue.

In another advantageous configuration, the cell processor according to the invention is equipped with a device for discharging substances or active ingredients to a defined human or animal tissue. Such a device is, for example, a device for generating an electromagnetic field which is applied in the use of magnetized nanoparticles. Here, the nanoparticles are pulled from the immune cell by a magnetic field applied with the help of the device. However, the discharge of the substance can be realized not only with the help of a magnetic field generated by the implantable microcell processor, but also by a magnetic field generated outside or minimally invasively within the body. Thus, the substance or the active ingredient is discharged as desired locally to the tissue. Analogous to the loading of the cells, here static and/or oscillating magnetic fields come into question. Through suitable selection of the field form and strength, the active ingredient can be fed to the desired tissue in a controlled way and thus ideally dosed. In another embodiment, the device for discharging substances or active ingredients is a device for destroying modified cells at the location of the desired tissue. Such a device can be, for example, a chemical to which the substance or the active ingredient dissolving the cell at the desired tissue was added. Triggers of the self-destruction can be, for example, the concentration of messenger materials, which are segregated by the desired tissue. Another possible configuration of the device for discharging substances or active ingredients is a device for generating ultrasound fields with a field strength sufficient for destroying the cells. The ultrasound field for discharging the substance can be generated not only as described by the implantable microprocessor itself, but the substance can also be discharged by an ultrasound field generated outside of the body or minimally invasively within the body.

Another advantageous configuration of the cell processor according to the invention is equipped with a device for localizing modified cells, for example, in the human body. The device for localizing can be, for example, a sensor for detecting a magnetic field generated by modified cells or a detecting device for biomarkers or a detecting device for fluorescent light.

Another advantageous configuration of the cell processor according to the invention concerns the integration or the arrangement of a rechargeable battery for supplying power. If the cell processor is used outside of the body, then advantageously a conventional rechargeable battery power supply can be fallen back upon. If the cell processor is used within the body, it is advantageous to use a long-service-life rechargeable battery, like those also used, for example, in cardiac pacemakers (lithium-ion rechargeable batteries). Such a long-service-life rechargeable battery can be exchanged through a minimally invasive operation. In another advantageous embodiment, the cell processor according to the invention is equipped with a contactless, inductive power supply. Here, for example, at the location of the implanted cell processor, a first coil with a rectifier is inserted, which powers a rechargeable battery or a capacitor, such as a SCAP [switched capacitor analysis program], which, in turn, represents the power supply for the cell processor. A SCAP is a high-power double-layer capacitor wherein the electrical energy is stored by charge shifting at the boundary between the electrode—usually made from carbon—and the organic electrolytes. The capacitor or the rechargeable battery is charged inductively via the first coil by a second coil which is applied to the body surface and to which an alternating field is applied. This outer second coil can be fixed, for example by a band on the body, in the sleeping phase. In another possibility for the configuration of the cell processor according to the invention, power is supplied through the use of special carbon nanotubes. These carbon nanotubes generate an electric charge when carrying a flow of a fluid, for example, blood. Thus, the necessary energy is provided by the fluid flow.

In another advantageous embodiment of the cell processor, there is at least one reservoir on or attached to the cell processor. Such a reservoir is used for storing at least one substance or active ingredient. The substances or active ingredients can be, for example, therapeutic agents, such as medicines, medicine precursors (prodrugs), hormones, enzymes for cleaning medicine precursors, viruses which are used, for example, for gene therapy, or nanoparticles. If the reservoirs are emptied in the case of an implanted cell processor, they can be refilled, for example, from the outside through a minimally invasive operation, e.g., with a fine needle. The filling can be realized by various kinds of septa. Another advantageous configuration of the cell processor according to the invention concerns the integration of a so-called home monitoring system, which reports, with a transmitter, the necessity for applying a substance or active ingredient.

In one advantageous embodiment, the cell processor or parts of the cell processor according to the invention are composed of biocompatible material and/or different types of metal, such as, silver, titanium, or V2A, and/or ceramic and/or plastics, such as polyethylene, silicon, polymer 908. Here, the surface of the cell processor is advantageously modified such that it is not identified by the immune system as a foreign body, or the cell processor secretes substances which suppress local defense reactions of the body (e.g., steroids).

The cell processor described above for modifying cells distinguishes itself through a series of considerable advantages. It allows the targeted transport of active ingredients of a wide variety with the help of the body's natural immune defense, such as T-lymphocytes, monocytes, or neutrophils (the latter after stimulation, for example, with β-glucan; see Hong et al., 2003, Cancer Research 63; 9023-9031) or other blood cells. In this way, the blood cells are held by the cell processor and substances, active ingredients, or therapeutic means such as medicines, medicine precursors (prodrugs), hormones, enzymes for cleaving the medicine precursors, viruses that are used, e.g., for gene therapy, or nanoparticles are transmitted to these cells. The defense cells modified in this way are then fed back into the human or animal bloodstream. The body's natural immune defense has the natural ability to recognize certain tissues. Through targeted treatment measures, a tissue can also be made recognizable to the immune system, i.e., target immune cells can be directed to a certain tissue and the recognition of this target tissue can be reinforced by immune cells.

One example is the targeted heating of a tumor, e.g., through hyperthermia or thermotherapy.

In this way, so-called heat shock proteins are produced in the heated tissue, such as, for example, HSP 96, HSP 72. These can play a role in the complex process leading to the antigen presentation at the cell surface. Furthermore, they can also be discharged in the extracellular environment and used for the immune system as signs for abnormal, dead, or damaged cells. An immune response in this tissue is reinforced.

An immune therapy with specific or multi-tumor-specific epitopes represents another example, or induction of the immune response through infiltration of cells or effector cells presenting corresponding antigens in the immune system, which also increase the reinforcement of the immune system in the tumor tissue. In addition, DNA vaccination is also conceivable in addition to the administration of protein antigens.

By reinforcing the immune response in the target tissue through prior treatment, e.g., heating before or during the use of the biocell processor or cell processor, the medicine transport also becomes more specific and more effective through loaded immune cells.

Immunization with the preparation of heat shock proteins (HPS) which are directed toward cancer cells was described as a stimulating factor for the immune system with regard to cancer identification (Blanchere et al. 1997).

Thus with the modified cells, with the help of the natural ability of the body's natural immune defense, certain tissue can be controlled in a targeted way and the active ingredient can be discharged at the desired position. The type of transmission of the active ingredient on the transport medium with the help of the cell processor according to the invention can vary as described. Both an addition (adsorption) of the active ingredient on the surface and also a storage (absorption) in the transport medium are possible. Also, the type of active ingredient addition can vary as described. Among other things, mechanisms such as electroporation, lipofection, or microinjection are possible. The cell processor according to the invention can transmit the active ingredients to the transport medium both within the body and outside of the body.

The transport of the medicine or the other components through the immune cells can be realized such that the material to be transported is stored in the immune cell or in another membrane, such as, e.g., a liposome, or is coupled to its surface (in the latter case, the material is not stored directly in the cell, but instead encapsulated in the other membrane, stored in the cell, or introduced into the cell added to the surface of this membrane.

The therapy according to the invention can be combined with all other therapies such as, e.g., cancer vaccination, reinforcement of the body's natural immune defense through, e.g., cytokine production, etc. The combination of therapies can be realized independent of each other, but they can also happen in parallel, before, or after the other therapies.

It is also conceivable that the therapy according to the invention is connected to other therapies, such that, e.g., through the reinforcement of the body's natural immune defense or cancer vaccinations, the cells necessary for the desired purpose are first increased and then separated out of the bloodstream for the therapy according to the invention.

In the therapy according to the invention, with the help of the body's natural immune defense, medicines coupled with an antibody which recognizes the cell to be treated can also be transported.

An antibody which docks the defense cell to the cell to be treated in a targeted way can be coupled to the body's natural defense cell.

With the help of the defense cell, in addition to medicines, all other materials, such as, e.g., nanoparticles, viruses, liposomes, proteins, nucleic acids, amino acids, hormones, radioactive particles, antibodies, antigens, peptides, all types of chemicals, etc., can also be transported.

As an example application, e.g., synthetic or natural hereditary information, e.g., RNA (e.g., siRNA) or DNA can be transported, which is linked to the cell to be treated or to its hereditary information, whereby the cell, e.g., is made harmless, further growth or cell division is prevented, or else is made more recognizable for the immune system and therefore cells of the immune system are directed in a targeted way to the treated cells (for example, cancer cells).

Cell processors according to the invention for modifying human or animal cells can be configured as described in one of the following examples.

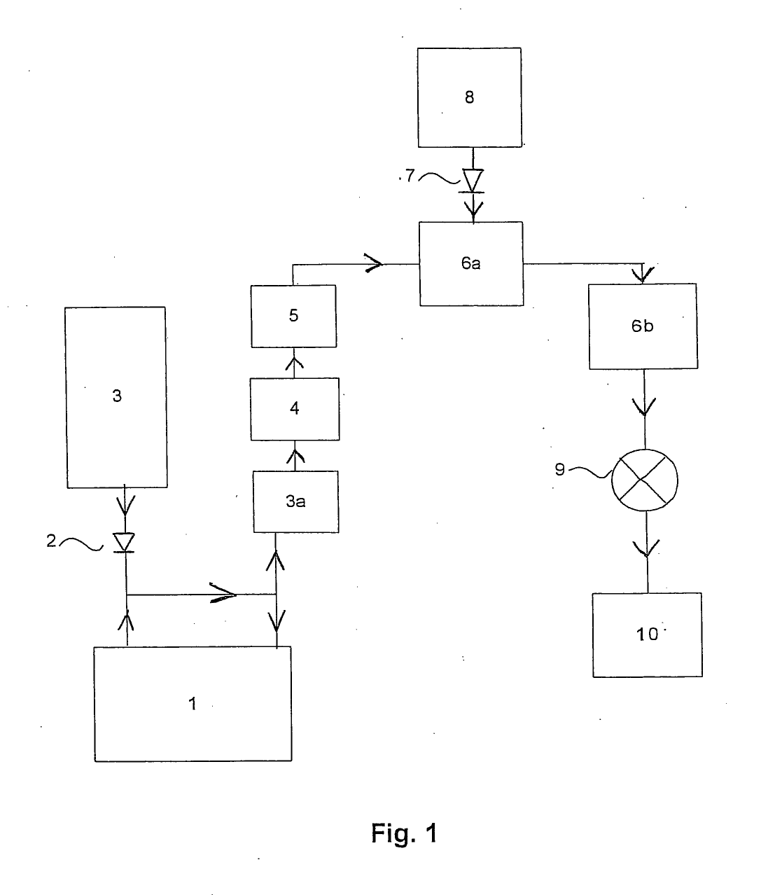

FIG. 1 shows schematically a cell processor according to the invention,

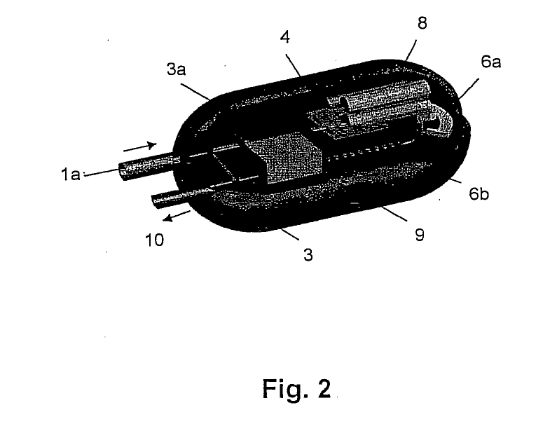

FIG. 2 shows a three-dimensional view of the same,

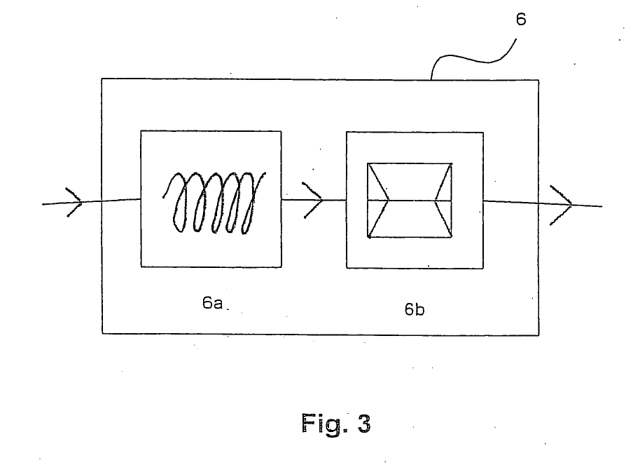

FIG. 3 shows devices for active ingredient loading and for active ingredient concentration measurement in a common reaction chamber,

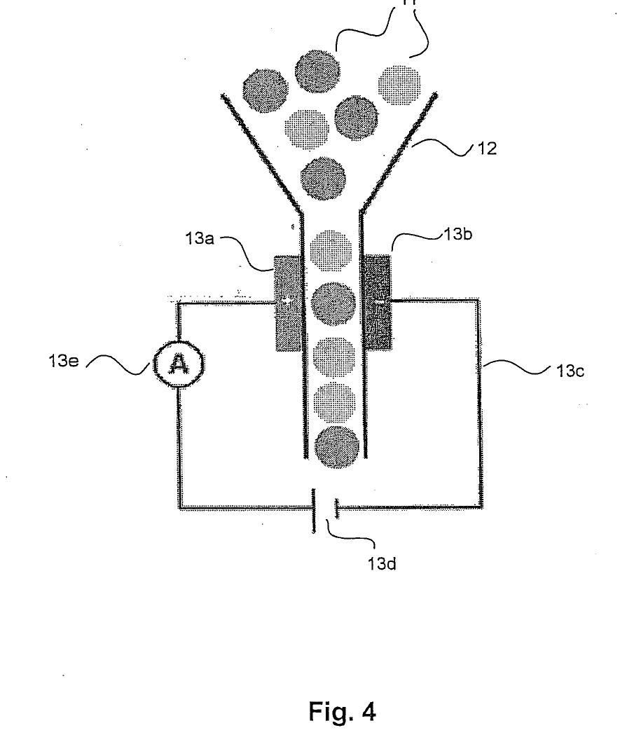

FIG. 4 shows a component device of the cell processor for cell isolation through measurement of the conductivity,

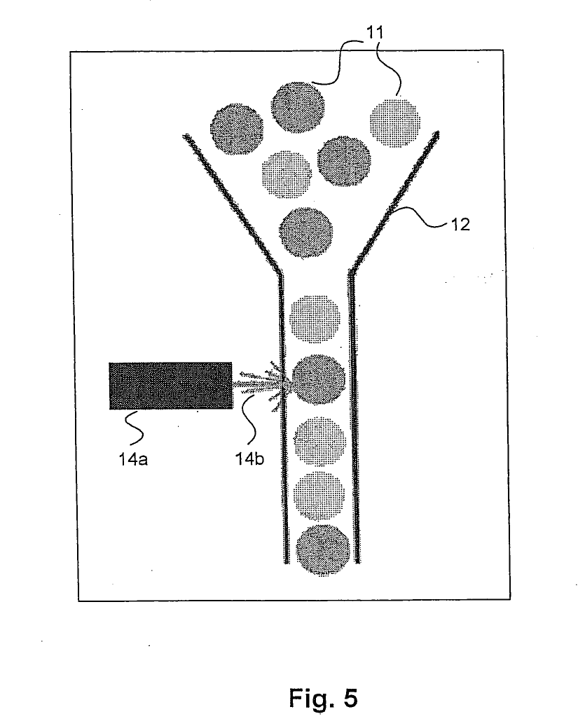

FIG. 5 shows a component device for cell isolation by means of a laser detector,

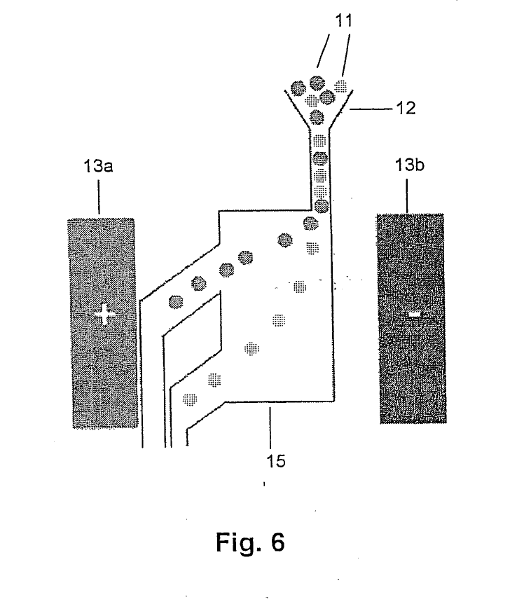

FIG. 6 shows a component device for cell isolation by means of the particle charge,

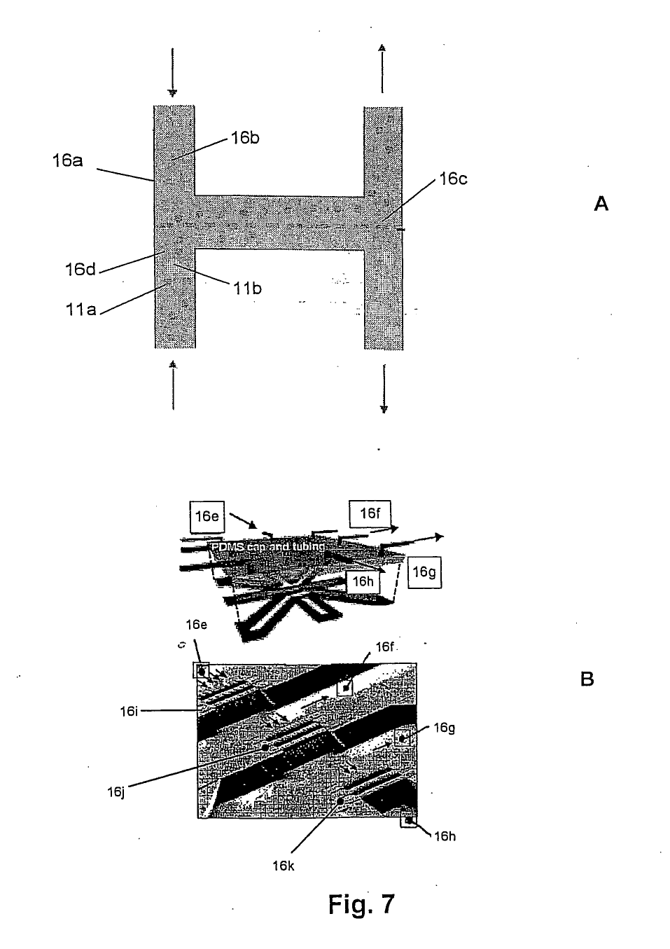

FIG. 7 shows component devices for cell isolation with the help of filters,

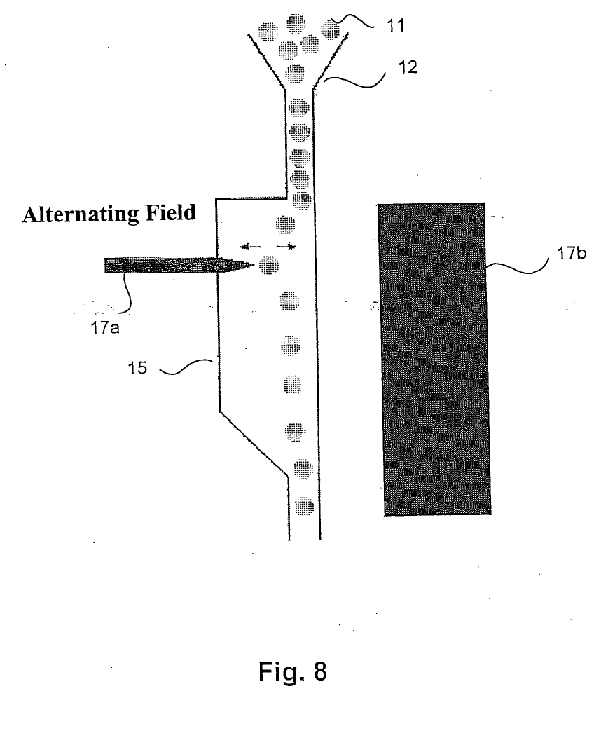

FIG. 8 shows a component device for cell fixing by means of an alternating field,

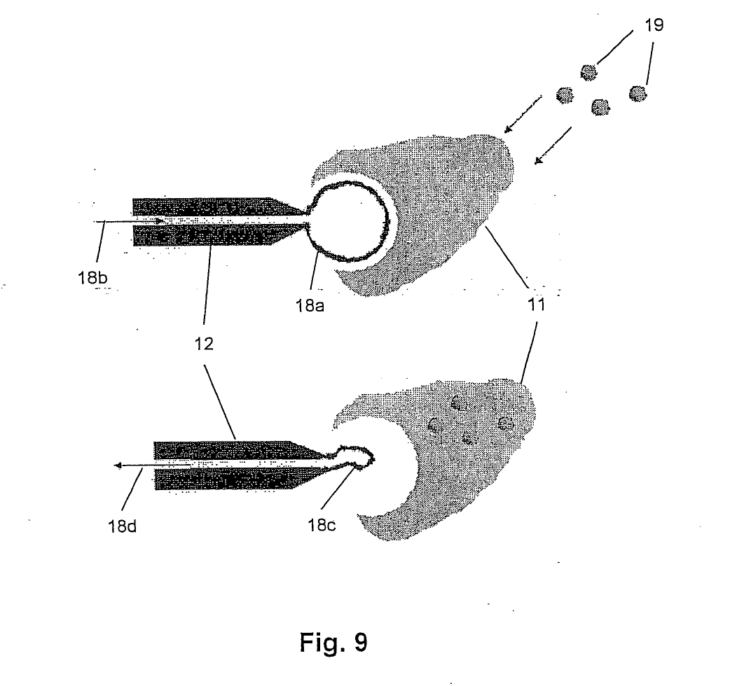

FIG. 9 shows a component device for cell fixing with the help of a microball,

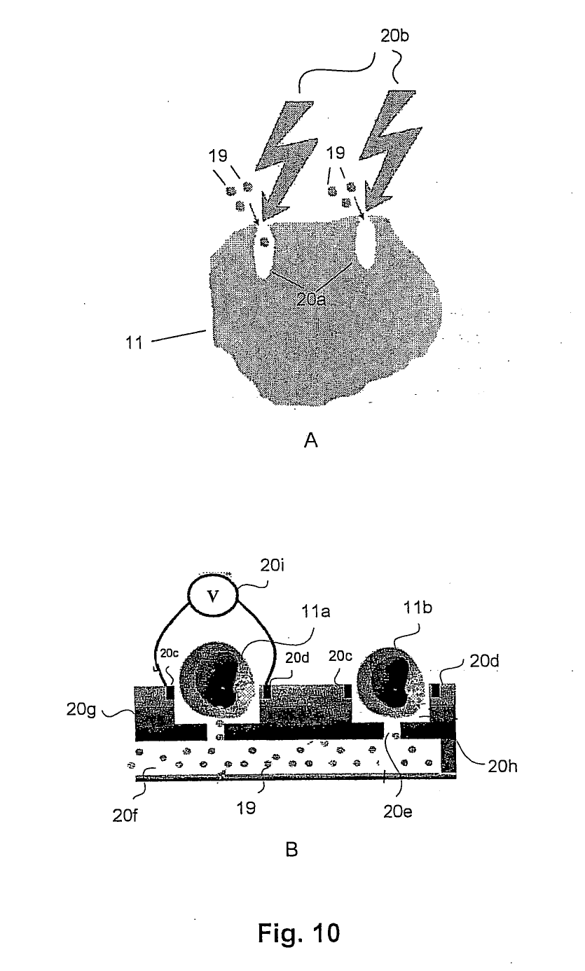

FIG. 10 shows the electroporation of a cell,

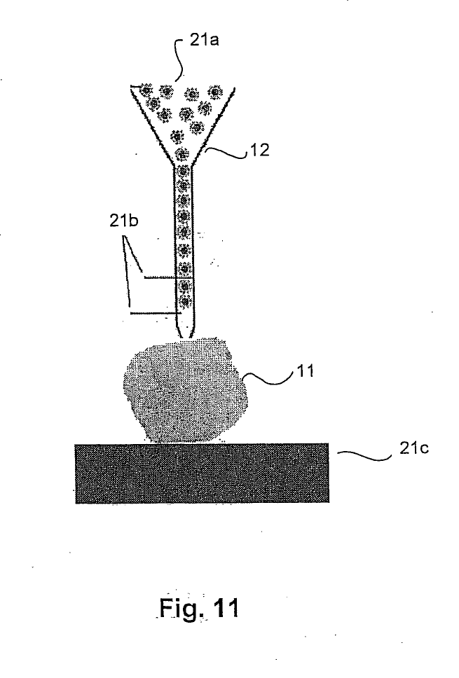

FIG. 11 shows a component device for loading cells by means of magnetized particles,

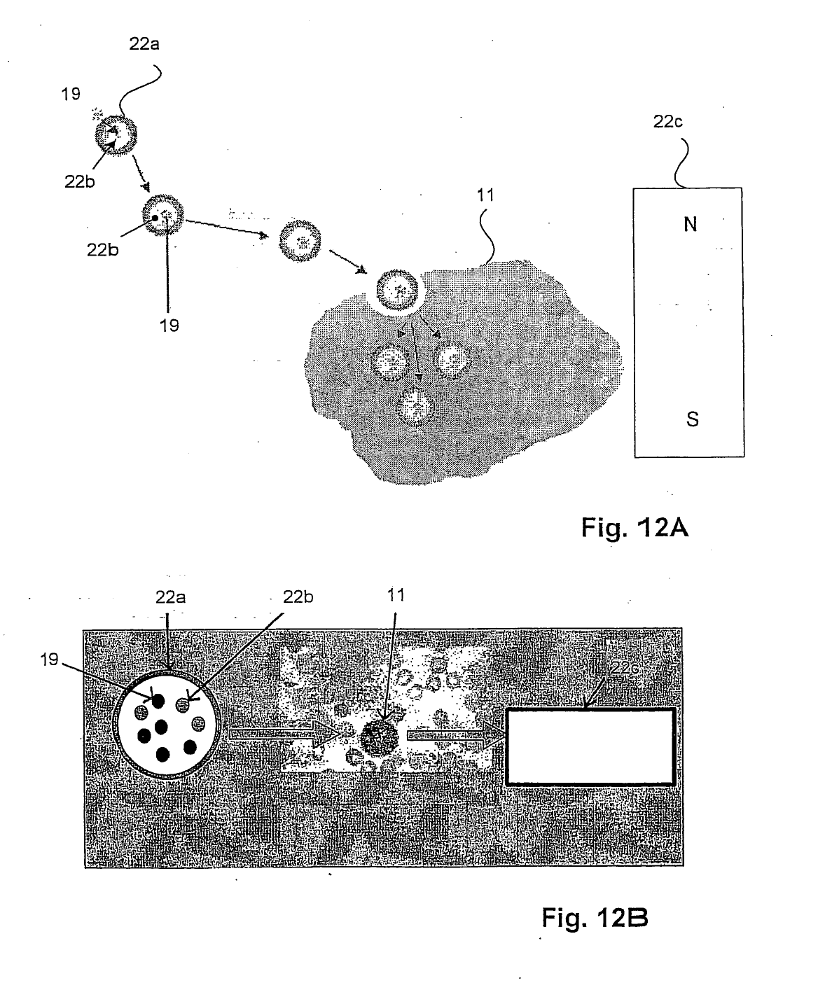

FIG. 12 shows the loading a cell by means of lipofection,

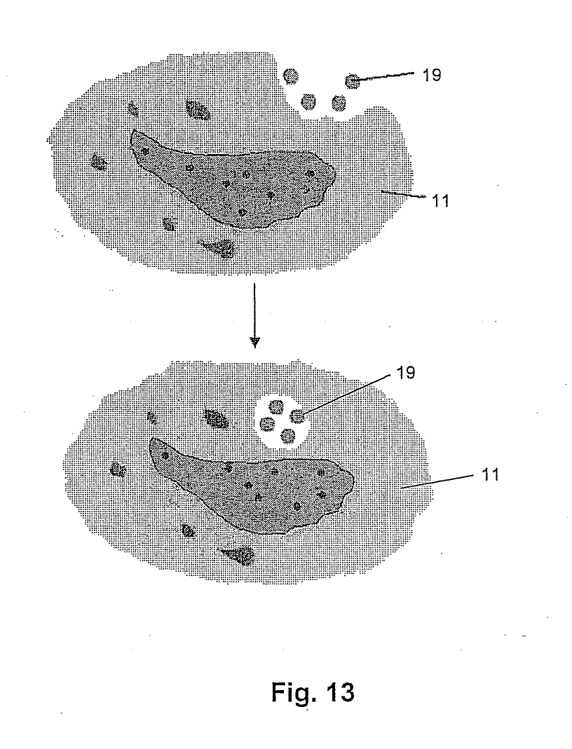

FIG. 13 shows the loading of a cell through phagocytosis,

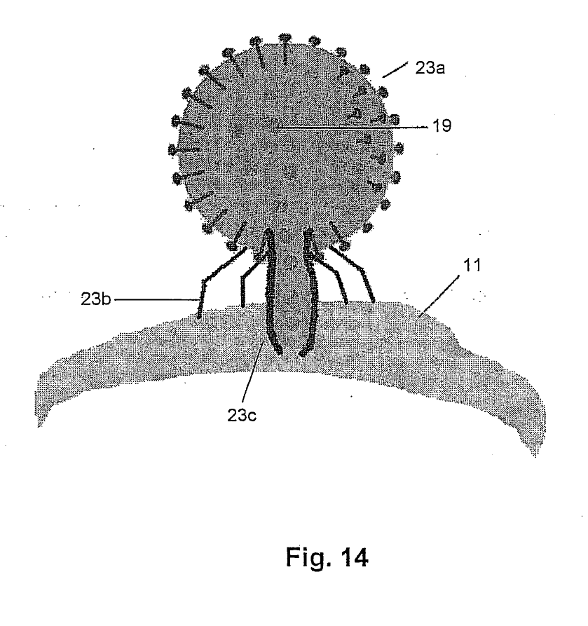

FIG. 14 shows the loading of a cell with the help of a virus,

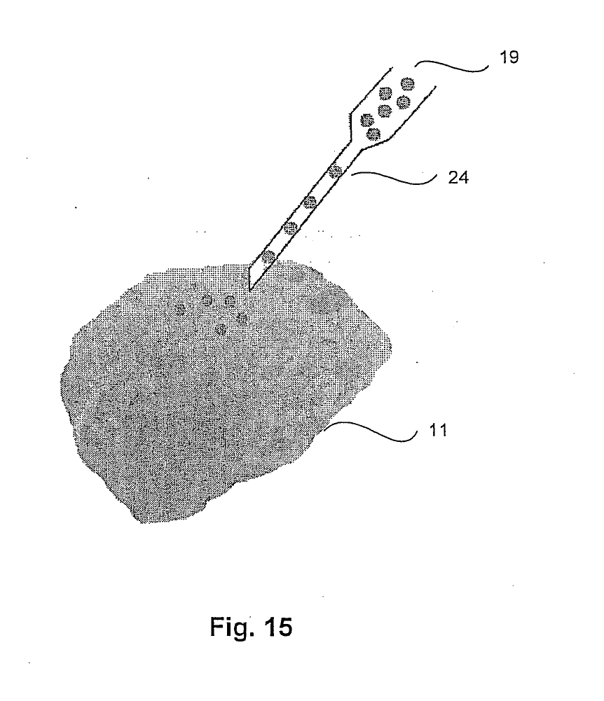

FIG. 15 shows the loading of a cell through microinjection,

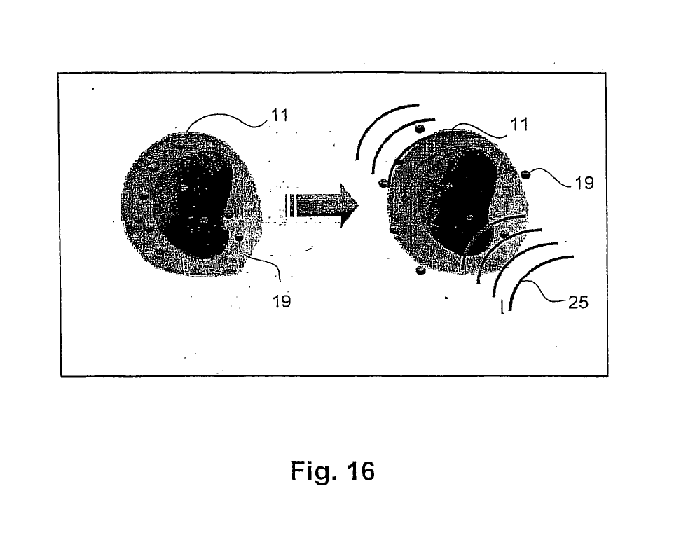

FIG. 16 shows the unloading of the active ingredient from a cell with the help of an ultrasound field,

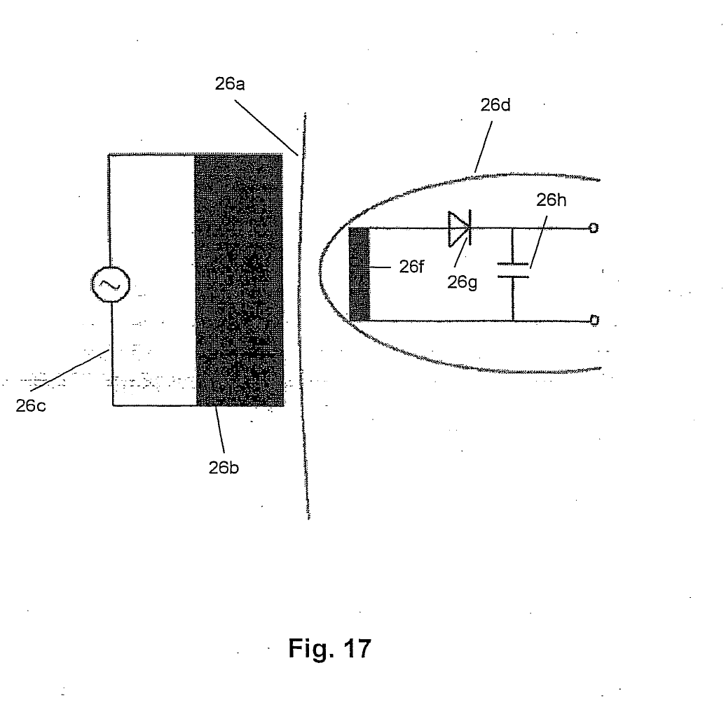

FIG. 17 shows an inductive power supply of the cell processor,

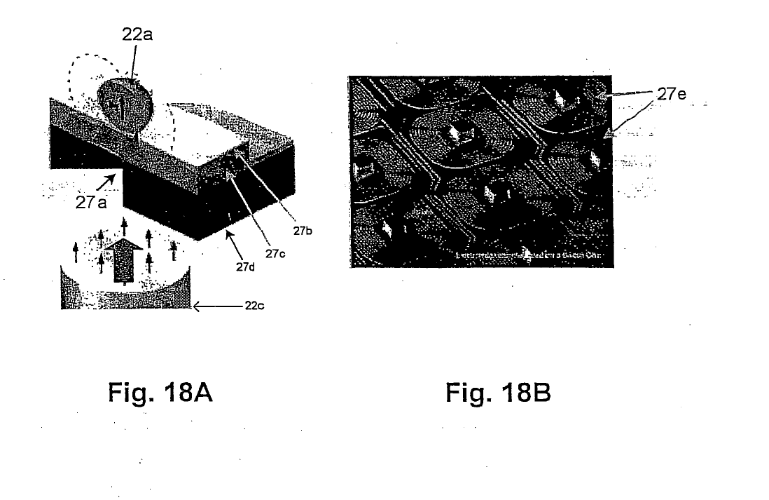

FIG. 18 shows a magnetoresistive sensor, as well as an arrangement of microcoils, and

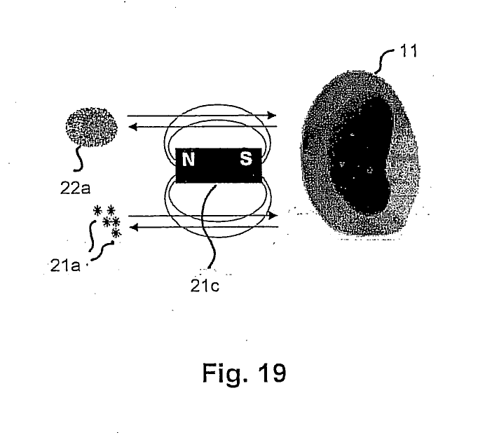

FIG. 19 shows the active ingredient loading and unloading with the help of magnetic liposomes or magnetic nanoparticles.

In the figures described below, the same reference symbols are used for the same or corresponding components or parts of the cell processor.

FIG. 1 schematically shows a possible configuration of a cell processor according to the invention. From a blood reservoir 1, the human bloodstream, blood is fed via a filtering device 3a to the cell isolation device 4, which selects the desired cells, i.e., the cells to be modified. In this way, a filter fluid from a filter fluid reservoir 3 can be added via a microvalve 2. The quantity of isolated cells is determined with the help of a counting device 5, a device for impedance measurement. In a loading device 6a, the cells are fixed and loaded with the desired active ingredient. The active ingredient is added via a lock 7 from an active ingredient reservoir 8. In the measurement device 6b, the concentration of the active ingredient in the loaded cells is determined. Through an outlet 10, the modified cells are fed to the human bloodstream. The cells are transported with the help of a micropump 9.

FIG. 2 shows a three-dimensional view of one embodiment of the cell processor according to the invention. Through an inlet 1a, bloods flows with its cellular components into the microcell processor. Via a filter structure 3a, the cells are guided to the cell isolation device 4. With the help of the component device 6a, the cells are loaded with the desired active ingredient, which is fed with the help of a reservoir 8. The concentration of the active ingredient introduced is measured with the help of the device for determining the concentration of the active ingredient 6b. Via the outlet 10, the modified cells are fed back into the bloodstream. The cell transport within the system is realized with the help of a micropump 9. The device 3 represents a tank for a filter fluid.

FIG. 3 shows an integrated device 6 for fixing the cells and for loading the cells with active ingredients with the help of magnetic fields generated by microcoils 6a and for measuring the concentration of active ingredients in the loaded cells by means of a magnetoresistive sensor 6b.

FIG. 4 shows a device for isolating cells with reference to their conductivity. The figure shows a capillary 12 with an end expanding upward like a funnel. In this expanded end and in the narrow part of the capillary 12, there are cells 11. At the narrow part of the capillary 12 are electrodes 13a and 13b respectively at the left and right; the electrodes are connected to a voltage source 13d. If a cell is led through the intermediate space between the two electrodes 13a and 13b then, depending on their conductivity, a current that is measured with a detecting device, such as an ammeter 13e, flows in the circuit 13c. With reference to the measured current, various cell types can be differentiated dependent on the conductivity of the cells 11; thus the desired cells are isolated.

FIG. 5 shows a device for cell isolation by means of a laser detector. Analogous to FIG. 4, a capillary 12 with an end expanding upward like a funnel and also cells 11 are shown. Instead of the electrodes arranged in FIG. 1, a laser detector 14a is arranged at the narrow left side of the capillary 12. With the help of a laser beam 14b, the light refraction properties of the cells 11 are determined. Because different cell types differ in their light refraction properties, the cell types can be differentiated with the help of the described device. In this way, the desired cell types are isolated.

FIG. 6 shows a device for cell isolation with reference to the particle charge of the cells. Analogous to FIG. 4, a capillary 12 with an end expanding upward like a funnel is shown. At the bottom end of the capillary 12, a widening 15 is attached. In the funnel-shaped end, in the narrow part, and in the widening of the capillary 12, cells 11 are shown. Two electrodes 13a and 13b are shown to the left and right of the widening. With the help of the electrodes 13a and 13b, an electromagnetic field is applied in the region of the widening 15. Due to the different particle charges, different cell types feature different propagation times in the electromagnetic field. With reference to this propagation time, the different cell types can be differentiated or the desired cell type can be isolated.

FIGS. 7A and 7B show devices for isolating cells with the help of filter modules. In FIG. 7A, an H-shaped filter module 16a is shown. In the center of the crossbar of the H there is a filter membrane 16c which divides the H-shaped filter module 16a into two equal size sections, an upper section and a lower section. In the lower section there is a solvent 16d, in which all of the cell types are dissolved. In the case shown, these include a desired cell type 11a and also another cell type 11b. In the upper part there is a solvent 16b which is suitable only for the desired cell type 11a. Thus the desired cell type 11a has the tendency to diffuse through the membrane 16c, while all of the other cell types or components do not. In the case shown, the cell isolation or selection is performed with the help of a filter membrane 16c and also two suitably selected solvents 16b and 16d.

FIG. 7B shows a multistage filter process. The cells are fed through an inlet channel 16e of the filter device. In the filter device, three filters 16i, 16j, and 16k are integrated. These three filters are used for separating particles with different diameters. Thus, after filtering with filter 16i, cells of a first diameter are discharged through a channel 16f; after filtering with filter 16j, cells of a second diameter are discharged via a channel 16g; and after filtering with filter 16k, cells are discharged via a third channel 16h.

FIG. 8 shows a device for cell fixing by means of an alternating electric field. A capillary 12 is shown with an end expanding upward like a funnel and also a widening 15 attached at the bottom. Cells 11 are shown over the entire area of the capillary. A needle 17a is inserted from the left into the widening. With the help of a second electrode 17b, an electric field is applied to the needle. Due to the particle charge, the cells 11 can be held with the help of the needle 17a. To hold the cells 11 over a long time period, the particle charge must be maintained. For this purpose, the cells 11 must be subject to a certain motion. Therefore, in the present case, an alternating electric field is used which alternately holds and then releases a cell 11 so that its oscillates and thus the particle charge is maintained.

FIG. 9 shows a device for fixing the cells with the help of a microball. In the upper part of the figure there is a capillary 12, on whose right end a microball 18a is attached. The microball is inflated, which happens with the help of a gas or a fluid 18b guided through the capillary. Through the inflation, a foreign body is simulated. A defense cell 11 surrounds this foreign body. In this way, the defense cell 11 is fixed and an active ingredient 19 is introduced. In the lower part of the figure, it is analogously shown how the fluid or the gas is removed from the microball 18d, the microball thus collapses 18c, and the defense cell 11, in which the active ingredient 19 has been inserted, swims free again.

FIG. 10 shows how the introduction of an active ingredient into a cell is performed with the help of high-voltage pulses, so-called electroporation. FIG. 10A shows an immune cell 11 on whose top part reversible pores 20a are formed in the cell membrane through high-voltage pulses 20b. Through these pores 20a, an active ingredient 19 is introduced into the immune cell 11. FIG. 10B shows a corresponding device for electroporation. Two cells 11a and 11b are immobilized on a plate 20g equipped with corresponding recesses 20h. On the left and right edge of the recesses 20h there are electrodes 20c and 20d. A voltage source 20i, which is shown for cell 11a, is connected to these electrodes. Underneath the plate there is a reservoir 20f, which is filled with the active ingredient 19 to be introduced. After the cell membrane pores are opened with the help of high-voltage pulses, the active ingredient 19 is introduced into the pores via microholes 20e underneath the recesses 20h.

FIG. 11 shows a device for loading a cell with an active ingredient by means of magnetized nanoparticles. In the top part of the figure, a capillary 12 with a top end expanding like a funnel is shown. In the bottom part, in the narrow part, the capillary 12 has a lock device 21b. The top end of the capillary 12, expanding like a funnel, is used as a reservoir for nanoparticles 21a. Underneath the capillary there is an immune cell 11, under this a magnet 21c. The nanoparticles 21a are loaded or coated with the desired active ingredients. By means of the magnet 21c, these particles are attracted. The lock device 21b has the effect that only a certain defined number of nanoparticles 21a can always pass the lock. With the help of the magnetic field, this defined number of nanoparticles with the active ingredients is drawn into the immune cell 11. For this device, the deciding factors are the time and the strength of the magnetic field, so that the nanoparticles 21a do not completely pass the immune cell 11, but instead remain in it. As magnetic fields, for example, static and oscillating fields are possible. As nanoparticles 21a, for example, Fe₃O₄ particles or paramagnetic particles can be used. With the help of the particles, the active ingredient is brought into a form which makes it indigestible for the immune cell. This is described in more detail later (in FIG. 13).

FIG. 12 shows the introduction of an active ingredient into an immune cell 11 with the help of lipofection. Circular liposomes 22a are shown in FIG. 12A. These liposomes are loaded with the desired active ingredient 19 and with magnetized particles 22b. In the right part of FIG. 12A is an immune cell 11, in whose interior there are four liposomes 22a. The liposomes 22a have the ability to penetrate the cell membrane of an immune cell 11 and thus to transport the active ingredient 19 inserted in the liposomes into the cell 11. In the example shown, the liposomes are directed toward the cell 11 by the magnetized particles 22b inserted in them by means of a magnet 22c. This is shown in FIG. 12A by arrows. FIG. 12B shows the determination of the concentration of an active ingredient into a cell processor according to the invention. The figure shows at the left a liposome 22a into which magnetic particles 22b and the active ingredient 19 have been introduced. The liposome is introduced into a cell 11 by means of lipofection, which is shown by an arrow. The concentration of the active ingredient is determined with the help of a magnetic sensor 22c. This is also shown by an arrow.

FIG. 13 shows the loading of an immune cell 11 with an active ingredient 19 by phagocytosis. In the top part of the figure, an immune cell 11 and also an active ingredient 19 are shown. In the bottom area of the figure, this same immune cell 11 is shown, but which has now completely surrounded the active ingredient 19. In phagocytosis, the natural function of an immune cell 11 is used. The active ingredient 19 is recognized as a foreign body and engulfed by the immune cell 11. Beforehand, the active ingredient 11 was brought into a form which makes it indigestible for the immune cell 11. It can be made indigestible, e.g., by applying or encasing the active ingredient by means of an outer capsule or through special formulation of the active ingredient.

Therefore, the cell itself is also protected from the effect of the medicine.

The capsule or formulation has properties through which it is triggered at the target location, e.g., through external heat supply at the target or through triggering of certain physiological conditions.

Therefore, the medicine displays the effect only at the target location. The medicine can also represent a precursor (prodrug), which transitions into the active form only at the target location.

FIG. 14 shows the loading of an immune cell 11 with an active ingredient with the help of a modified HIV virus 23a. In the top part of the drawing, a virus 23a is shown in the form of a sectioned sphere. This virus 23a contains in its interior the active ingredient 19. In the bottom area of the figure, an immune cell 11 is shown. The virus 23a adheres to the immune cell 11 with the help of four leg-like projections 23b. On the bottom side, the virus 23a has a blowpipe-like projection 23c, which projects into the immune cell 11. In the case shown, the virus 23a is used to introduce the active ingredient 19 into the immune cell 11. Alternatively, the virus 23a can also be used to introduce DNA into the cell 11, which causes the cell 11 to produce the corresponding active ingredient 19.

FIG. 15 shows the loading of a cell 11 with an active ingredient through microinjection. The figures shows an immune cell 11, a needle 24, and also an active ingredient 19 which is located partially in the needle 24 and partially already in the immune cell 11. In the present example, the active ingredient 19 is injected by microinjection with a very fine needle 24 into the immune cell 11.

FIG. 16 shows the release of an active ingredient 19 on the desired tissue with the help of ultrasound energy. At the left in the figure, a cell 11 loaded with an active ingredient 19 is shown. After this has traveled into the desired tissue, the active ingredient 19 is released from the cell 11 with the help of an ultrasound field 25 of sufficient intensity, shown at the right in the figure.

FIG. 17 shows a device for inductive power supply for the cell processor. In the left part of the figure, a first coil 26b with associated power supply 26c is shown in the form of a rectangle. In the right half of the figure, an implant 26d which contains a cell processor is shown as an ellipse. Between the first coil 26b and the implant 26d is the skin surface 26a. In the implant 26d, there is a second coil 26f, a rectifier 26g, and a capacitor or rechargeable battery 26h. The power supply of the cell processor happens as follows: an alternating field is applied to the coil 26b. Through the inductive effect, a current which powers the rechargeable battery or the capacitor 26h is produced in the coil 26f.

FIG. 18 shows the determination of the concentration of an introduced active ingredient with the help of a magnetoresistive sensor. In FIG. 18A, an active-ingredient-loaded liposome 22a which contains magnetic particles is drawn in the form of a sectioned sphere. Underneath the liposome 22a, a magnetoresistive sensor 27a is shown in a three-dimensional view. This essentially consists of three layers, the top Si₃N₄ layer 27b, the magnetoresistive film 27c underneath, and also a silicon substrate 27d underneath. An electromagnet 22c is shown under the magnetoresistive sensor 27a. The position of the liposome 22a can be affected with the help of the electromagnet 22c. The concentration of the active ingredient loaded into the liposome 22a is determined by the magnetoresistive sensor 27a with the help of the magnetic field generated by the magnetic particles. FIG. 18b shows an arrangement of microcoils 27e for generating or for detecting a magnetic field.

FIG. 19 shows the loading and the unloading of a cell with the help of magnetic fields. At the top left in the figure is shown a liposome 22a which is loaded with an active ingredient and with magnetic particles. Underneath, a few magnetic particles 21a are shown. In the center of the figure, a magnet 21c is shown with its magnetic field. At the right in the figure, a cell 11 is shown. With the help of the magnet 21c, the magnetized liposome 22a can be influenced in its position or in its path. This is shown by two arrows. With the help of the magnet 21c, it is not only possible to direct the active ingredient carrier (liposome) in the direction of the cell, but it is also possible to detect the dispersion of active-ingredient-loaded immune cells in the human body. The magnetic nanoparticles 21a can also be supplied to the cell 11 with the help of the magnet 21c or its magnetic field. With the help of the magnet 21c, unloading of the active-ingredient-coated particles 21a from the cell 11 is also possible. This is shown by two arrows.

1. Cell modification device for modifying human or animal cells for the purpose of a therapeutic effect in the human or animal body by the modified cells, wherein the device contains at least one device for isolating the cells and at least one device for introducing at least one substance into the cells and/or for arranging at least one substance on the cells or is composed of the devices mentioned.

2. Device according to claim 1, wherein the device is implantable in the human or animal body or that the device can be used outside of the human or animal body, especially as a device that can be carried on the body or used as a laboratory instrument.

3. Cell modification device according to claim 1, wherein the device has at least one device for fixing the cells and/or at least one device for determining the concentration of at least one substance in or on the cells and/or for determining the number of modified cells.

4. Device according to claim 1, wherein the device has at least one device for introducing the cells into the human or animal bloodstream.

5. Device according to claim 1, wherein the device for introducing at least one substance into the cells and/or for arranging at least one substance on the cells and the device for determining the concentration of the one or more substances in or on the cells and/or for determining the number of modified cells are arranged in a common reaction chamber or in different compartments.

6. Device according to claim 1, wherein at least one device for transporting and/or regulating cell flows is integrated into the device or arranged on it.

7. Device according to claim 6, wherein the device for transporting and/or regulating cell flows contains at least one micropump and/or at least one microvalve and/or at least one micronozzle or is composed of these components.

8. Device according to claim 1, wherein the device has at least one device for monitoring the correct function of the individual devices.

9. Device according to claim 1, wherein the device has at least one device for detection and/or for counting and/or for determining the instantaneous stopping location of modified and/or non-modified cells.

10. Device according to claim 9, wherein the device contains the device for detection and/or for counting and/or for determining the instantaneous stopping location, a sensor for detecting a magnetic field, and/or a device for the identification of biomarkers and/or a device for detecting fluorescent light or is composed of these parts.

11. Device according to one of claim 1, wherein the device contains at least one device for generating a static and/or time-variable magnetic field.

12. Device according to claim 11, wherein the device for generating the magnetic field contains at least one microcoil and/or a permanent magnet or is composed of these parts.

13. Device according to claim 1, wherein the device has at least one device for introducing a washing solution.

14. Device according to claim 1, wherein the device is equipped with at least one device for discharging the substances to or in a defined human or animal tissue.

15. Device according to claim 14, wherein the device for discharging substances contains a device for generating a magnetic field or consists of these parts.

16. Device according to claim 15, wherein the device for generating the magnetic field contains at least one microcoil or consists of this part.

17. Device according to claim 14, wherein the device for discharging the substances contains a device for destroying the modified cells or consists of these parts.

18. Device according to claim 17, wherein the device for destroying the modified cells contains or is composed of a chemical, adding the substances for dissolving the modified cells at the location of the defined human or animal tissue.

19. (canceled)

20. Device according to claim 1, wherein at least one rechargeable battery especially a lithium-iodine rechargeable battery, and/or at least one battery for supplying power is integrated or arranged in or on the device.

21.-26. (canceled)

27. Device according to claim 1, wherein at least one reservoir is integrated or arranged in or on the device for storing the substances.

28. Device according to claim 27, wherein at least one reservoir is completely or partially filled with the substances.

29. Device according to claim 27, wherein at least one of the reservoirs is refillable.

30. Device according to claim 29, wherein at least one of the reservoirs has at least one septum for refilling.

31. Device according to claim 1, wherein at least one device for changing the substances is integrated into or attached to the device.

32. Device according to claim 1, wherein the substances contain or are composed of active ingredients, medicines, medicine precursors (prodrugs), hormones, enzymes, viruses, nanoparticles, DNA or DNA parts, and/or substances for local suppression of the defense reaction of the animal or human body.

33.-34. (canceled)

35. Device according to claim 1, wherein the material of the device or parts of the device contains or is composed of metal and/or metal alloys, especially comprising or consisting of titanium and/or silver and/or V2A steel, and/or ceramic and/or plastic, especially comprising or consisting of polyethylene and/or silicon and/or polymer 908, and/or a biocompatible substance and/or chemically inert substance and/or substances that do not interact with cells.

36.-37. (canceled)

38. Device according to claim 1, wherein the device has a surface which is not recognizable as a foreign body by the human or animal immune system.

39. Device according to claim 1, wherein the device for isolating the cells contains or is composed of a device for isolating cells with the help of their electrophoretic mobility and/or has at least one capillary for conducting cells, and at least two electrodes and arranged on at least one of the capillaries for determining the conductivity of the cells and/or for measuring the impedance of the cells.

40. Device according to claim 39, wherein, instead of or in addition to the electrodes, there is a detector system containing at least one laser on at least one of the capillaries for determining the light refraction of the cells.

41. Device according to claim 1, wherein the device for isolating the cells has at least one capillary for conducting cells and that the end of at least one capillary has a widening and/or that on its end a widening is arranged, and that a device for generating an electromagnetic field is arranged inside or integrated into the widening on the device for determining different propagation times of differently-loaded cells.

42. Device according to claim 41, wherein the device for generating an electromagnetic field contains or is composed of at least two electrodes.

43.-49. (canceled)

50. Device according to claim 1, wherein the device for introducing at least one substance into the cells and/or for arranging at least one substance on the cells contains or is composed of a device for generating high-voltage pulses.

51. Device according to claim 50, wherein step-shaped high-voltage pulses can be generated with the help of the device for generating high-voltage pulses.

52. Device according to claim 50, wherein the device for generating high-voltage pulses contains or is composed of at least two electrodes.

53. (canceled)

54. Device according to claim 1, wherein the device for introducing at least one substance into the cells and/or for arranging at least one substance on the cells contains or is composed of magnetized or magnetizable particles, wherein the particles are coated with or contain the substance.

55. Device according to claim 54, wherein the particles have sizes in the range from over 1 nm and/or below 10⁴ nm, especially from over 3 nm and/or below 1000 nm.

56. Device according to claim 54, wherein the particles contain or are composed of Fe₂O₃ and/or Fe₃O₄ and/or paramagnetic substances.

57. Device according to claim 54, wherein the device for introducing at least one substance into the cells and/or for arranging at least one substance on the cells contains or is composed of at least one reservoir for the particles and/or at least one capillary, to or in which at least one lock device for controlling the number of passing particles is attached or integrated, and/or at least one device for generating a magnetic field.

58. Device according to claim 57, wherein the device for generating a magnetic field contains or is composed of at least one microcoil.

59. Device according to claim 57, wherein a magnetic field with a static and/or a time-varying field portion can be generated with the help of the device for generating a magnetic field.

60. Device according to claim 1, wherein the device for introducing at least one substance into the cells and/or for arranging at least one substance on the cells contains or consists of liposomes.

61. Device according to claim 60, wherein the liposomes contain magnetic particles.

62. Device according to claim 1, wherein the device for introducing at least one substance into the cells and/or for arranging at least one substance on the cells contains at least one device for modifying the substance such that this substance cannot be changed by immune cells.

63. Device according to claim 1, wherein the device for introducing at least one substance into the cells and/or for arranging at least one substance on the cells contains or consists of viruses.

64. Device according to claim 63, wherein the viruses are modified HIV viruses.

65. (canceled)

66. Device according to claim 1, wherein the device for determining the concentration of at least one substance in or on the cells contains or consists of a device for determining the strength of a magnetic field and/or a device for detecting fluorescent light and/or a device for detecting biomarkers.

67. (canceled)

68. Device according to claim 1, wherein the one or more devices for introducing the cells into the human or animal bloodstream contain or consists of at least one micropump.

69. Cell modification method for modifying human or animal cells outside of the human or animal body, wherein the cells are isolated in a first step and that then at least one substance is introduced into the cells and/or arranged on the cells.

70. Cell modification method for modifying human or animal cells inside the human or animal body, wherein the cells are isolated in a first step and that then at least one substance is introduced into the cells and/or arranged on the cells.

71. Method according to claim 69, wherein a device according to claim 1 is used.

72. Method according to claim 69, wherein the cells are held after isolation and before introduction and/or placement of the substance and/or that after introduction and/or placement of the substance, the concentration of the one or more substances is measured in and/or on the cells and/or that the number of modified cells is determined.

73. Method according to claim 72, wherein after determining the concentration of the one or more substances in and/or on the cells and/or the number of modified cells, the one or more substances is subjected to secondary processing and a washing agent is introduced.

74. Method according to claim 69, wherein the electrical conductivity of the cells is determined and the cells are isolated with the help of the determined conductivity.

75. Method according to claim 69, wherein the light refraction through the cells is determined and the cells are isolated with the help of the determined light refraction.

76. Method according to claim 69, wherein the propagation time of the cells in a magnetic and/or electric field is determined and the cells are isolated with the help of the determined propagation times.

77.-82. (canceled)

83. Method according to claim 69, wherein at least one of the substances is introduced into and/or arranged on the cell through particle bombardment.

84. Method according to claim 69, wherein at least one of the substances is applied to magnetized or magnetizable particles that the particles are optionally magnetized, and that the particles are introduced into the cells with the help of magnetic fields.

85. (canceled)

86. Method according to claim 84, wherein the concentration of at least one of the substances in or one the cells or the number of cells loaded with the substance is determined with the help of the magnetic field generated by the particles.

87. Method according to claim 69, wherein at least one of the substances is introduced in liposomes.

88. Method according to claim 87, wherein at least partially magnetic particles are introduced in the liposomes.

89. Method according to claim 84, wherein a static or time-varying magnetic field is used for influencing the stopping location of the cells.

90. Method according to claim 84, wherein the cells are localized with the help of the magnetic field of the particles.

91. Method according to claim 69, wherein at least one of the substances is provided in a form which makes it indigestible for the cells.

92. Method according to claim 69, wherein at least one of the substances is introduced with the help of viruses into the cells and/or arranged on the cells.

93.-94. (canceled)

95. Method according to claim 69, a fluorescing substance is applied to at least one of the substances and that the fluorescence is measured for determining the concentration of the one or more substances in and/or on the cells.

96. Method according to claim 69, wherein a biomarker is applied to at least one of the substances for determining the concentration of the one or more substances in and/or on the cells.

97. Use of a method according to claim 69 and a device according to claim 27, wherein the reservoirs are refillable, especially through a minimally invasive operation e.g. with at least one needle.

98. (canceled)

99. Use of a device and/or a method according to claim 1 for modifying human or animal cells outside or inside of the human and animal body or for modifying immune cells or cellular components of blood.

100.-101. (canceled)

102. Use according to claim 99 for the purpose of therapy for the human or animal body, for therapy of cancerous diseases especially tumor diseases or diseases of the brain, for therapy of cancerous diseases of the liver or for treating infectious and inflammatory diseases, arteriosclerosis, autoimmune diseases such as multiple sclerosis and other diseases, in which the cells control metastases in a targeted way.

103.-105. (canceled)
